(19) United States
(12) Patent Application Publication
(10) Pub. No.: US 2007/0150023 A1
(43) Pub. Date: Jun. 28, 2007

(54) Transvisceral neurostimulation mapping device and method

(76) Inventors: Anthony R. Ignagni, Oberlin, OH (US); Raymond P. Onders, Shaker Heights, OH (US); James E. Gelbke, North Royalton, OH (US)

Correspondence Address: SHAY LAW GROUP, LLP, 2755 CAMPUS DRIVE, SUITE 210, SAN MATEO, CA, 94403, US

(21) Appl. No.: 11/607,428

(22) Filed: Nov. 30, 2006

Related U.S. Application Data
(60) Provisional application No. 60/597,440, filed on Dec. 2, 2005.

(51) Int. Cl. A61N 1/00

(52) U.S. Cl. 607/42

(57) ABSTRACT

The invention provides a method and device for providing electrical stimulation to a patient's diaphragm (or other organ or tissue) including the steps of: introducing an endoscope transviscerally (e.g., transgastrically) into the patient's body cavity; delivering an electrode into the patient's body cavity through a lumen of the endoscope; applying suction to attach the electrode to a stimulation site on the diaphragm (or other organ or tissue); and delivering a stimulation pulse to the stimulation site. The stimulation may be repeated at multiple stimulation sites.

### CROSS-REFERENCE

This application claims the benefit under 35 U.S.C. § 119 of U.S. Patent Application No. 60/597,440 filed Dec. 2, 2005, and which is incorporated herein by reference in its entirety.

### BACKGROUND OF THE INVENTION

Electrodes are implanted into patients for a variety of purposes, such as to stimulate muscle movement and to provide pain relief. For example, U.S. Pat. Nos. 5,472,438 and 5,797,923 and U.S. Patent Appl. Publ. No. 2005/0021102 describe neurostimulation of a patient's diaphragm to assist the patient's breathing.

Correct placement of stimulation electrodes helps achieve the best results. For example, optimal neurostimulation of a patient's diaphragm requires placement of the stimulation electrode or electrodes at or near phrenic nerve motor points. As described in U.S. Patent Appl. Publ. No. 2005/0021102, the desired stimulation electrode placement may be determined via a mapping procedure in which a mapping electrode is temporarily placed on the diaphragm, a stimulus pulse is delivered and the magnitude of the diaphragm's response to the stimulation is measured. This mapping is repeated multiple times at different locations on the diaphragm so that the clinician may determine which stimulation locations provide the best muscle movement response (i.e., the phrenic nerve motor points). U.S. Pat. No. 5,472,438 and U.S. Patent Appl. Publ. No. 2005/0107860 describe neurostimulation electrode mapping tools that may be used to access and map the diaphragm laparoscopically.

### SUMMARY OF THE INVENTION

Laparoscopic neurostimulation electrode mapping requires at least two incisions in the patient's abdomen, one for viewing and one for delivery of the electrode tool. In addition, earlier neurostimulation mapping tools lacked the ability to mark stimulation locations, thus requiring the use of a separate marking tool. The invention provides a neurostimulation mapping device and method that minimizes abdominal incisions (and resulting scars) by using a transvisceral (e.g., translumenal) approach to the abdominal cavity.

One aspect of the invention provides a method of providing electrical stimulation to target tissue of a patient (such as the diaphragm or other organ or tissue) including the steps of: introducing an endoscope transviscerally (e.g., transgastrically) into a body cavity of the patient (such as the abdominal cavity); delivering an electrode into the patient's body cavity through a lumen of the endoscope; applying suction to attach the electrode to a stimulation site on the target tissue (such as the diaphragm or other organ or tissue); and delivering a stimulation pulse to the stimulation site. The stimulation may be repeated at multiple stimulation sites.

In some embodiments according of the invention in which the electrode is part of an electrode tool, the step of delivering the electrode includes the step of passing the electrode tool through the endoscope lumen and applying suction through a suction lumen of the electrode tool. In some embodiments, the electrode tool also has a handle, and the step of applying suction includes the step of actuating a suction actuator on the handle. Some embodiments include the step of releasing suction to detach the electrode, such as by actuating a release actuator on a handle of the electrode tool. In some embodiments, the step of delivering a stimulation pulse is performed by actuating a stimulating actuator on a handle of the electrode tool.

Some embodiments of the invention include the step of using the electrode tool to mark the stimulation site with a marking agent. For example, the electrode tool may have a marking port, and the step of using the electrode tool to mark the stimulation site may be performed by delivering a marking agent through the marking port, such as by actuating a marking actuator on a handle of the electrode tool.

Another aspect of the invention provides a method of providing electrical stimulation to target tissue within a patient including the steps of: introducing an endoscope transviscerally (e.g., transgastrically) into a body cavity of the patient; passing an electrode tool through the endoscope lumen, the electrode tool comprising an electrode and a marker; placing the electrode at a stimulation site on the target tissue(such as the diaphragm or other organ or tissue); delivering a stimulation pulse to the stimulation site; and marking the stimulation site with the electrode tool marker. The stimulation and marking may be repeated at multiple stimulation sites.

In some embodiments, the method includes the step of applying suction to the stimulation site through a suction lumen of the electrode tool after placing the electrode, such as by actuating a suction actuator on a handle of the electrode tool. Some embodiments include the step of releasing suction to detach the electrode, such as by actuating a release actuator on a handle of the electrode tool. In some embodiments, the step of delivering a stimulation pulse is performed by actuating a stimulation actuator on a handle of the electrode tool. In some embodiments, the marking step is performed by actuating a marking actuator on a handle of the electrode tool.

In some embodiments, the electrode tool marker includes a marking lumen and a marking agent port, and the marking step is performed by delivering a marking agent through the marking lumen and marking agent port.

Yet another aspect of the invention provides an endoscopic electrode tool having a body adapted to be inserted through a working channel of an endoscope transviscerally into a body cavity of the patient (such as the abdominal cavity) to a tissue stimulation site, with the body including a suction lumen and a suction port at a distal end of the body communicating with the suction lumen, and an electrode supported by the body at the distal end of the body, the electrode being connectable with a source of stimulation current.

In some embodiments, the electrode tool has a handle supporting a proximal end of the electrode tool body, the handle being adapted to advance and withdraw the electrode tool from an endoscope inserted translumenally into a patient's abdominal cavity. The handle may have a suction actuator adapted to apply suction to the suction lumen to attach the electrode to the stimulation site; a suction release actuator adapted to release suction from the suction lumen; and/or a stimulation actuator adapted to apply stimulation current from the stimulation source to the electrode. In some embodiments, the electrode tool body has a marking lumen communicating with a marking agent port at the distal end of the body, the marking lumen and marking agent port being adapted to deliver a marking agent to the stimulation site, and the handle may have a marking actuator adapted to deliver a marking agent through the marking lumen to the marking port.

In embodiments with a marking port, the electrode may surround the marking port. The electrode may also surround the suction port. The marking port, suction port and electrode may all be disposed on a lateral wall of the electrode tool body. Some embodiments may provide a plurality of suction ports at the distal end of the body, and the suction ports and electrode may be disposed on a lateral wall of the electrode tool body.

Still another aspect of the invention provides an endoscopic electrode tool with a body adapted to be inserted through a working channel of an endoscope transviscerally into a body cavity (such as a patient's abdominal cavity) to a tissue stimulation site, the body having a marking lumen communicating with a marking lumen port at a distal end of the body, and an electrode supported by the body at the distal end of the body, the electrode being connectable with a source of stimulation current.

In some embodiments, the electrode tool has a handle supporting a proximal end of the electrode tool body, the handle being adapted to advance and withdraw the electrode tool from an endoscope inserted transviscerally into a patient's body cavity (such as the abdominal cavity). The handle may have a stimulation actuator adapted to apply stimulation current from the stimulation source to the electrode and/or a marking actuator adapted to deliver a marking agent through the marking lumen to the marking port. In some embodiments, the electrode surrounds the marking port.

### Incorporation by Reference

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flow chart showing an aspect of a tissue mapping method of this invention.

FIG. 2 shows an endoscope passing into a peritoneal cavity through an opening in a stomach.

FIG. 3 shows an endoscope and mapping instrument passing into a peritoneal cavity through an opening in a stomach and retroflexed toward a diaphragm.

FIG. 4 shows an endoscope and mapping instrument passing into a peritoneal cavity through an opening in a stomach.

FIG. 5 is a flowchart showing another aspect of the transgastric mapping and electrode placement methods of this invention.

FIGS. 6A-E are schematic drawings showing a transgastric procedure according to an aspect of this invention.

FIG. 7 is a partial cross-sectional drawing showing the distal end an electrode tool for use with the mapping device and method of this invention.

FIG. 8 is a partial cross-sectional drawing showing the distal end or an alternative electrode tool for use with a mapping device and method of this invention.

FIG. 9 is a cross-section of the electrode tool of FIG. 8.

FIG. 10 is a cross-section of an alternative electrode tool of this invention.

FIG. 11 shows a handle for use with an electrode tool of this invention.

### DETAILED DESCRIPTION OF THE INVENTION

The invention will be described with reference to transgastric mapping of a patient's diaphragm as a prelude to electrode implantation for diaphragm neurostimulation. It should be understood, however, that the invention is generally applicable to other transvisceral access techniques, other target stimulation sites and other electrical stimulation purposes.

Mechanical ventilation via a tracheostomy is standard therapy for patients with tetraplegia after complete cervical spine injury above cervical level 3 (C3) and common among those with complete injuries at C4-C8. According to the 2005 NSCISC Database 21.2% (2,503) of all individuals with tetraplegia and 7.1% (748) of all individuals with paraplegia required a mechanical ventilator for pulmonary support during their initial rehabilitation admission. At the time of rehabilitation discharge 7.1% (748) of all individuals with tetraplegia and 0.7% (75) of all individuals with paraplegia required a mechanical ventilator for pulmonary support. The proportion of persons with tetraplegia who required the use of mechanical ventilation also increased from 13.9% prior to 1980 to 32.1% between 1990 and 1994. Yet this treatment is not without harm. Among patients with spinal cord injury at similar levels, the need for mechanical ventilation decreases survival rates from 84% in the non-ventilated group to only 33% in the ventilated group. Life expectancy among patients with SCI and mechanical ventilation is also decreased. Patients aged 20 years at the time of SCI have life expectancies of an additional 33-38 years as tetraplegics (mortality at 53-58 years of age), compared to a typical life expectancy of 58 additional years in a noninjured person of the same age (mortality at 78 years of age). With mechanical ventilation, life expectancy is decreased even further to only 23.8 additional years (mortality at 44 years of age). The need for mechanical ventilation affects older persons to an even greater extent; the 45-year-old SCI person on a ventilator has a life expectancy of only 8.9 additional years (www.spinalcord.uab.edu, 2004). Use of diaphragm pacing stimulation helps avoid the greatest risk of mortality to these patients: pneumonia introduced by the ventilator circuit.

Similarly, the greatest risk of death in amyotrophic lateral sclerosis (ALS) patients is respiratory failure and pulmonary complications, accounting for at least 84% of deaths. ALS afflicts approximately 6,000 new patients every year in the U.S. with a 3-5 year survival and no known cure. The only treatment currently approved by the FDA is Rilutek which has demonstrated a modest three month improvement in survival. Respiratory deterioration is usually gradual and, although the major cause of death, rarely leads to the diagnoses.

The placement of a percutaneous endoscopic gastrostomy (PEG) tube is common in trauma patients and ALS patients. In the PEG procedure, an endoscope is placed in the patient's stomach, and the stomach is insufflated to push the stomach wall against the abdominal wall. Light from the endoscope shining through the stomach wall guides the insertion of a needle and guidewire through the abdominal wall into the stomach. The guidewire is snared and pulled proximally through the patient's mouth. The guidewire is then used to pull the feeding tube through the patient's mouth into the stomach and through the openings in the stomach wall and abdominal wall until one end of the tube is in the stomach and the other is above the exterior surface of the patient's abdominal wall. The PEG tube can then be used to introduce liquid nutrients into the patient's stomach. PEG tube placement is standard of care for ALS patients and is typically accepted in up to 20% of such patients. Early PEG tube placement can lead to significantly lower mortality rates for these patients.

One embodiment of the invention relates to the use of transgastric diaphragm neurostimulation mapping in ALS patients or other patients who could benefit from both diaphragm stimulation and PEG tube feeding. Aspects of transgastric access of the inferior diaphragm or other abdominal structures may be found in U.S. patent application Ser. No. 11/467,014. It should be understood, however, that the diaphragm mapping and stimulation aspects of the invention may be used in patients who will not be receiving PEG tubes.

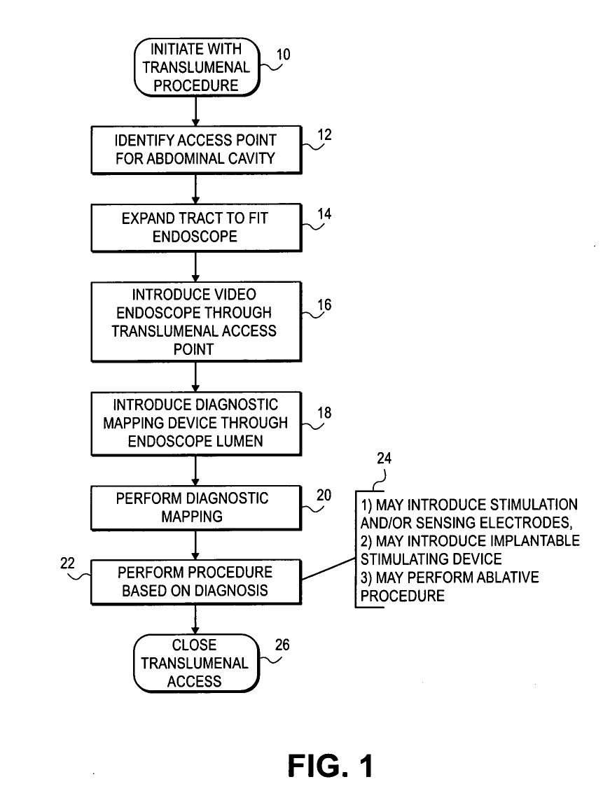

FIG. 1 is a flow chart showing an aspect of a tissue mapping method of this invention. The procedure initiates by placing an endoscope into the patient's stomach to provide translumenal access to the stomach wall, as in block 10 in FIG. 1. Using the endoscope's viewing capabilities, a peritoneal cavity access point in the stomach wall is identified (12). For example, one desirable section of stomach for this procedure may be located as far distally as is accessible by the endoscope, in a location that provides good visualization of the target abdominal or pelvic structures and that permits ready closing with a closing device.

After an opening is made in the stomach wall using a standard technique (e.g., gastrostomy), the opening is expanded to accommodate the endoscope (14), and the distal end of the endoscope is passed through the opening into the peritoneal cavity (16). After using the endoscope's viewing capabilities to locate target tissue site, a diagnostic mapping device is passed through a lumen of the endoscope so that its distal end is in the peritoneal cavity (18). Diagnostic electrical mapping may be then be performed on the target tissue (20)1: The mapping procedure may be used to diagnose the patient and to determine which therapeutic procedure should be performed, such as the implantation of stimulation or sensing electrodes, implantation of a stimulating device and/or tissue ablation (22, 24).

After completion of the procedure, the opening in the stomach is closed, and the endoscope is removed from the patient (26). Gastrostomy closing may be performed by placement of a percutaneous endoscopic gastrostomy (PEG) tube or by use of a ligating system, clip, T-bar device, or other device to close the opening without placement of a PEG.

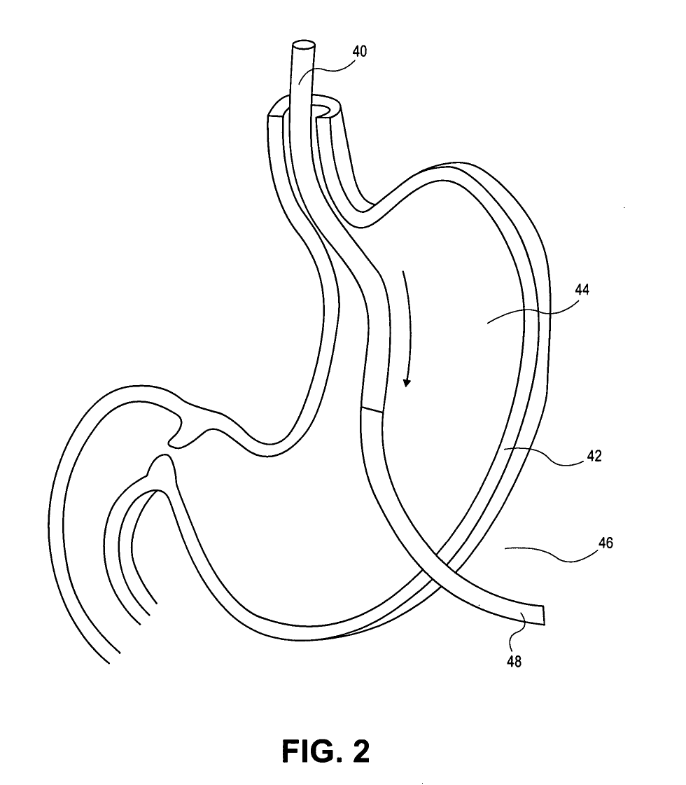

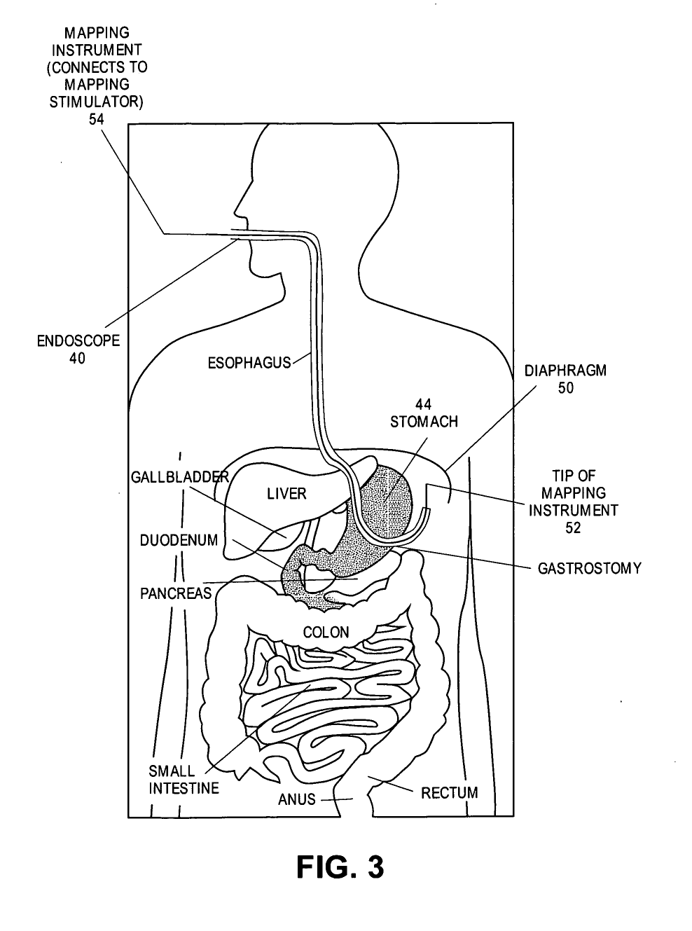

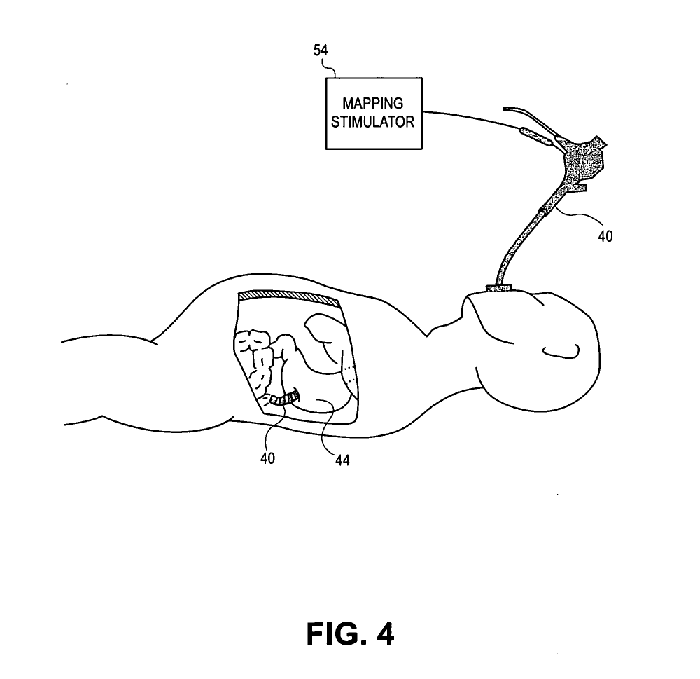

FIGS. 2-4 show an endoscope 40 passing into and through the wall 42 of a stomach 44 into the peritoneal cavity 46. The distal end 48 of the endoscope 40 may be retroflexed to view and/or provide access to, e.g., the patient's diaphragm 50, as shown in FIG. 3, which shows a mapping electrode 52 at the tip of a mapping instrument near the diaphragm. Other organs within and around the peritoneal cavity may be accessed, as shown. FIG. 4 shows how an external mapping stimulator may be connected with a mapping instrument 54. Other details regarding the formation of a gastrostomy, endoscopic access to the peritoneal cavity through a gastrostomy, and tissue mapping and stimulation in general may be found in U.S. Pat. No. 6,918,871; U.S. Patent Appl. Publ. No. 2004/0260245; U.S. Patent Appl. Publ. No. 2005/0277945; U.S. Patent Appl. Publ. No. 2001/0049497; U.S. Patent Appl. Publ. No. 2005/0021102; and U.S. Patent Appl. Publ. No. 2005/0107860.

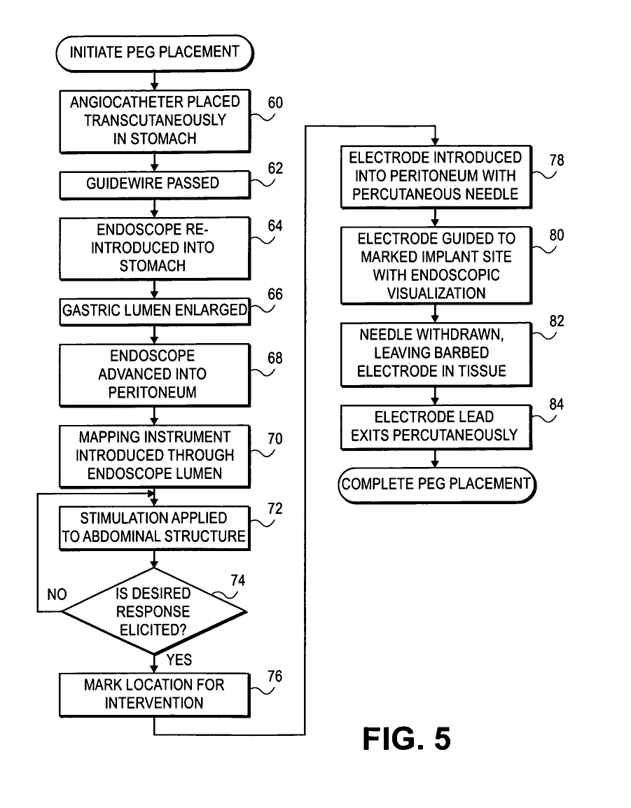

FIG. 5 is a flowchart showing another aspect of the transgastric mapping and electrode placement methods of this invention. A percutaneous endoscopic gastrostomy procedure commences by placing an angiocatheter percutaneously in the patient's stomach (60). A guidewire is then passed into the stomach (62), and an endoscope is introduced (or re-introduced) into the stomach (64). The guidewire may be snared by the endoscope and pulled out of the patient's mouth, and a second guidewire may be introduced with the first guidewire to provide a guide for re-introduction of the endoscope. An overtube may also be provided with the endoscope upon re-introduction. The gastric lumen or opening formed by the angiocatheter placement is enlarged, such as with a dilating balloon passed down the guidewire (66), and the distal tip of the endoscope is advanced through the opening into the patient's peritoneum (surrounding the peritoneal cavity) (68). The second guidewire and dilating balloon may then be removed.

After movement of the endoscope (e.g., bending, retroflexing) for visualization of target structures, a mapping instrument such as an electrode tool may be passed through a lumen of the endoscope to stimulate and map target tissue within the peritoneal cavity (70, 72). Mapping stimulation responses may be monitored with instrumentation (e.g., EMG, ENG, pressure catheters, etc.) or queried from the patient (as in the case of awake endoscopy for identifying sources of chronic pain). The mapping stimulation may be a single pulse to evoke a twitch or action potential or a train of pulses to elicit a contraction or propagation of nervous system impulses. If the desired response is not elicited in the target tissue, the mapping stimulation may be repeated (74). Otherwise, if mapping is successful, the target site may be marked for electrode placement or other intervention (76).

A stimulation electrode may then introduced into the peritoneum and placed in the target tissue, such as by a percutaneous needle under visualization from the endoscope (78, 80, 82). For example, an electrode such as a barbed style electrode (e.g., a Synapse Peterson, Memberg or single helix electrode) may be loaded into a non-coring needle and penetrated through the skin. Using endoscopic visualization and (if desirable or necessary) with an endoscopic grasping tool, the electrode may be placed in the target tissue. The needle may then be removed, leaving the electrode leads extending percutaneously for connection to an external stimulation device (84). Alternatively, barbed electrodes may be placed endoscopically by introducing a small gauge needle through a lumen of the endoscope for direct placement in the target tissue. The electrode leads may be connected to a subcutaneously-placed stimulator or to a microstimulator (such as a BION® microstimulator) passed through the endoscope lumen and placed with the electrode. As yet another alternative, the electrode may be placed laparoscopically using a single laparoscopic port and visualization from the endoscope. This alternative may permit the manipulation and placement of larger electrodes in the peritoneal cavity.

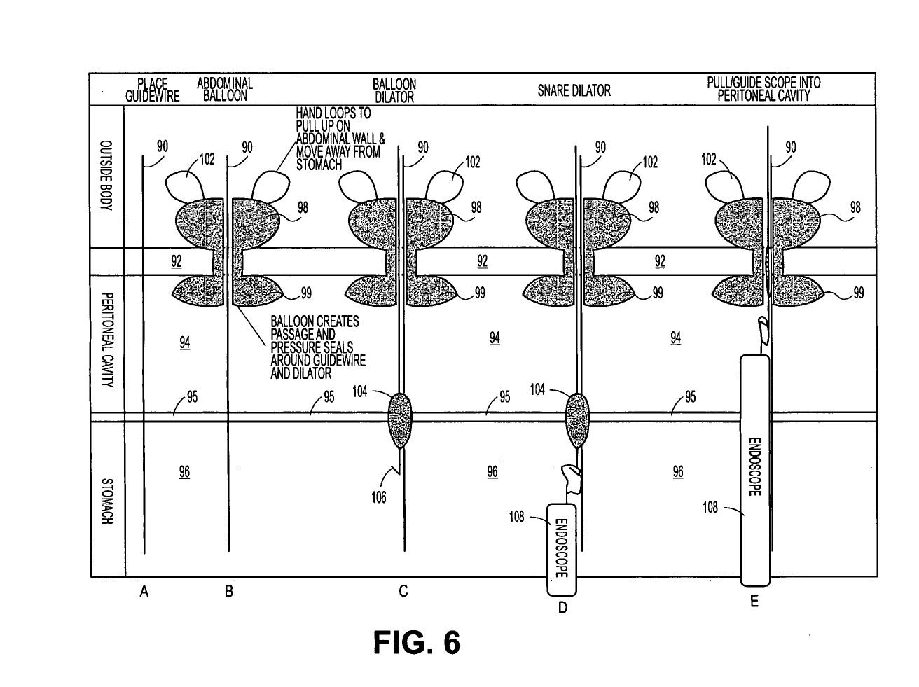

6A-E show schematically some of steps of endoscopic transgastric access of the peritoneal cavity according to one aspect of the invention. In FIG. 6A, a guidewire 90 is inserted percutaneously through the patient's abdominal wall 92, through the peritoneal cavity 94 and into the patient's stomach 96. A grasping device formed as a balloon 98 with a port 100 is placed around guidewire 90 and inflated to provide a pressure seal around the guidewire, as shown in FIG. 6B. An attachment portion 99 of balloon 98 extends through the abdominal wall 92, as shown, to firmly attach the grasping device to the abdominal wall. Balloon 98 has grasping elements formed as loops 102 that may be grasped by a user's fingers to pull the abdominal wall 92 away from the stomach during the procedure. A dilator 104 is advanced in a deflated configuration through the stomach wall 95 over guidewire 90, then inflated to enlarge the stomach wall opening, as shown in FIG. 6C. A snare 106 extending from dilator 104 grasps the distal end of endoscope 108 to pull endoscope 108 into the peritoneal cavity, as shown in FIGS. 6D and 6E. Use of the grasping loops 102 to pull the abdominal wall 92 away from stomach 96 is particularly useful during this portion of the procedure. Dilator 104 may be deflated, and snare 106 unhooked from endoscope 108, to permit endoscope 108 to be used in the peritoneal cavity as described above.

In some embodiments, the electrode tool has a contact electrode (formed, e.g., from stainless steel) supported by a flexible body. In some embodiments, the electrode tool has a suction port communicating with a vacuum source, and in some embodiments the electrode tool has a tissue marker, such as a port for delivering a marking agent to the tissue. The diameter of the contact electrode is constrained by the diameter of the endoscope working channel, such as 2.8 mm or 3.7 mm. The length and surface area of the contact electrode may be approximately the same as that of the stimulating electrode to be implanted after mapping, for example, a length of 9 mm and a surface area of 11 mm². The electrode tool should have an overall length permitting it to extend from outside the patient through the entire length of the endoscope (103 cm or 168 cm, for standard length endoscopes) and into the abdominal cavity. The electrode tool body should be flexible enough to prevent any damping of the diaphragm tissue response to the stimulus but stiff enough to maintain the patency of its suction lumen when vacuum is applied.

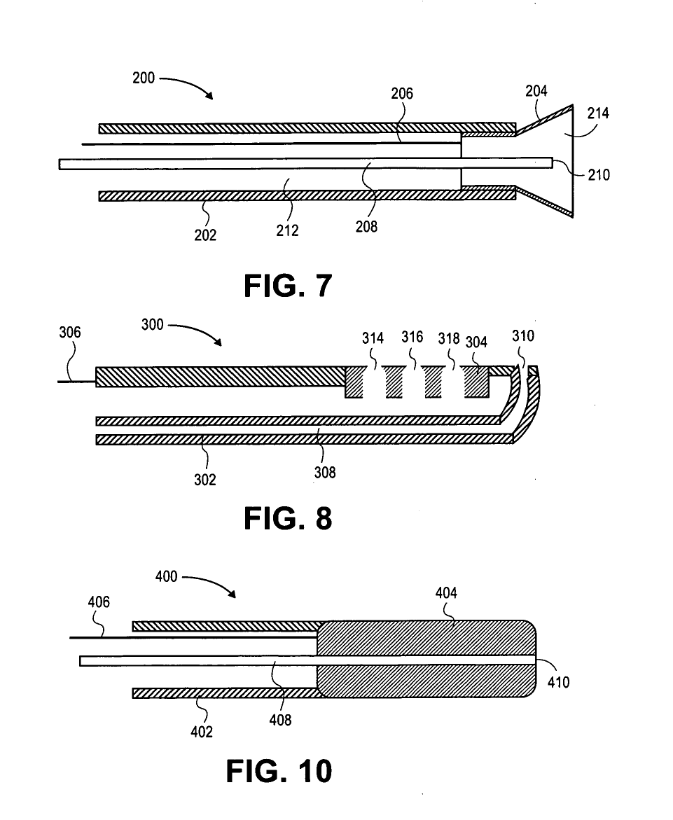

FIG. 7 shows the distal end an electrode tool 200 for use with the mapping device and method of this invention. Tool 200 has a body 202 (formed, e.g., from reinforced silicone tubing with a durometer of approximately 50) supporting an electrode 204 at its distal end. A lightweight metal coil may be added to the electrode tool body to provide sufficient support. Electrode 204 may be formed from a flared hypotube section. A wire 206 extends proximally from electrode 204 to the mapping instrument (not shown), optionally through a separate wire lumen. A marking lumen 208 extends proximally from a marking port 210 to a source of a marking agent (not shown). An annular suction lumen 212 surrounding marking lumen 208 and marking port 210 extends proximally from suction port 214 within electrode 204 to a vacuum or suction source (not shown).

In use, an endoscope is advanced transgastrically into the abdominal cavity as described above, and the electrode tool 200 is advanced through a working channel of the endoscope to place electrode 204 against the patient's diaphragm at a stimulation site. Visualization from the endoscope aids in placement. After placing the electrode, suction is applied through suction lumen 212 to hold the electrode in place, and a stimulus is applied (e.g., stimulus amplitude of 20 mA and pulse duration of 100 μs). The magnitude of the evoked muscle response, visual confirmation of the contraction, and/or the change in pressure of the abdominal cavity are noted and recorded. The location of the stimulation site may then be marked by ejecting a marking agent (such as gentian violet or india ink) from marking port 210. Suction is then released, and the electrode is moved to another stimulation site, where the procedure is repeated. The response of the diaphragm to stimulation at the multiple stimulation sites may be mapped on a grid overlying the endoscope monitor. The magnitude of the evoked muscle response and the resultant change in pressure of the abdominal cavity can then be used to identify the optimal electrode implant site of each hemidiaphragm. The optimal site, which is typically the phrenic nerve motor point of the hemidiaphragm, is chosen as the site that elicits a diffuse contraction and the greatest magnitude of pressure change. Using the markings as a guide, a stimulation electrode is then implanted under endoscopic visualization at the optimal site in each hemidiaphragm using, e.g., the implant tool described in U.S. Pat. No. 5,797,923, or other technique as described above.

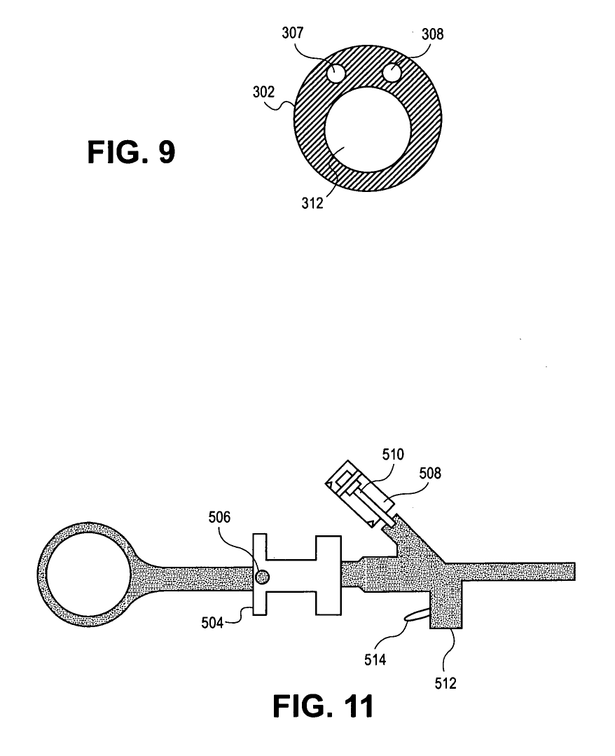

An alternative embodiment of an electrode tool 300 is shown in FIGS. 8 and 9. Tool 300 has a body 302 (formed, e.g., from reinforced silicone tubing with a durometer of approximately 50) supporting an electrode 304 on a side wall at its distal end. A wire 306 extends proximally from electrode 204 to the mapping instrument (not shown), optionally through a wire lumen 307. A marking lumen 308 extends proximally from a marking port 310 to a source of a marking agent (not shown). A suction lumen 312 extends proximally from suction ports 314, 316, and 318 within electrode 304 to a vacuum or suction source (not shown).

Use of the electrode tool 300 of FIG. 8 is similar to that of FIG. 7. Tool 300 is advanced transgastrically into the patient's abdominal cavity through an endoscope, and electrode 304 is placed against the patient's diaphragm. Suction is applied through suction lumen 312 to hold the electrode in place, and a stimulus is applied (e.g., stimulus amplitude of 20 mA and pulse duration of 100 μs). The magnitude of the evoked muscle response, visual confirmation of the contraction, and/or the change in pressure of the abdominal cavity are noted and recorded. The location of the stimulation site is then marked by ejecting a marking agent such as india ink from marking port 310. Suction is then released, and the electrode is moved to another stimulation site, where the procedure is repeated.

Yet another embodiment of the electrode tool is shown in FIG. 10. Unlike the earlier embodiments, the electrode tool 400 of FIG. 10 lacks a suction port. Electrode tool 400 therefore has a body 402 formed from a higher durometer tubing than the embodiments of FIGS. 7 and 8 so that the electrode 404 may be held in place on the diaphragm without suction. A wire 406 extends proximally from the electrode to the mapping instrument (not shown), optionally through a wire lumen. Marking ink may be delivered through a marking lumen 408 and marking port 410.

FIG. 11 shows a proximal handle for use with an electrode tool of this invention. Handle 500 extends proximally from the electrode tool body 502 and may be used to move and otherwise manipulate the tool from outside the patient. In addition, handle 500 has one or more actuators for operating the electrode tool. As shown, handle 500 has a suction actuator formed as a sliding piston 504 in sealed communication with the tool's suction lumen (not shown). Pulling piston 504 proximally (to the left, as shown in the figure) creates suction in the suction lumen. Ratchets, catches or other devices may be used to maintain the position of the piston after actuation. Handle 500 may also have a suction release actuator, such as release button 506 that releases the suction within the suction lumen by venting the suction lumen and/or permitting piston 504 to return toward its unactuated position. Handle 500 may also have a marking actuator, such as an ink reservoir 508 and ink ejector 510 (such as a plunger or a CO₂ charge) communicating with the tool's marking lumen (not shown). Handle 500 may also have an electrical connector 512 to connect the tool's electrode with a stimulus source (such as a surgical stimulator, not shown) as well as a switch 514 for operating the stimulus source.

### EXAMPLE 1

Methods: Pigs were anesthetized and transgastric peritoneal access with a flexible endoscope was obtained using a guidewire, needle knife cautery and balloon dilatation. The diaphragm was mapped to locate the motor point (where stimulation provides complete contraction of the diaphragm) with an endoscopic electrostimulation catheter. An intramuscular electrode was then placed at the motor point with a percutaneous needle. This was then attached to the diaphragm pacing system. The gastrotomy was managed with a gastrostomy tube.

Results: Four pigs were studied and the diaphragm could be mapped with the endoscopic mapping instrument to identify the motor point. In one animal, under trans-gastric endoscopic visualization a percutaneous electrode was placed into the motor point and the diaphragm could be paced in conjunction with mechanical ventilation.

Conclusion: These animal studies support the concept that transgastric mapping of the diaphragm and implantation of a percutaneous electrode for therapeutic diaphragmatic stimulation is feasible.

### EXAMPLE 2

Methods: Four female pigs (25 kg) were sedated and a single channel gastroscope was passed transgastrically into the peritoneal cavity. Pneumoperitoneum was achieved via a pressure insulator through a percutaneous, intraperitoneal 14-gauge catheter. Three other pressures were recorded via separate catheters. First, a 14-gauge percutaneous catheter passed intraperitoneally measured true intra-abdominal pressure. The second transducer was a 14-gauge tube attached to the endoscope used to measure endoscope tip pressure. The third pressure transducer was connected to the biopsy channel port of the endoscope. The abdomen was insulated to a range (10-30 mmHg) of pressures, and simultaneous pressures were recorded from all pressure sensors.

Results: Pressure correlation curves were developed for all animals across all intraperitoneal pressures (mean error −4.25 to −1 mmHg). Endoscope tip pressures correlated with biopsy channel pressures (R²=0.99). Biopsy channel and endoscope tip pressures fit a least-squares linear model to predict actual intra-abdominal pressure (R=0.99 for both). Both scope tip and biopsy channel port pressures were strongly correlative with true intra-abdominal pressures (R² =0.98, R²=0.99 respectively).

Conclusion: This study demonstrates that monitoring pressure through an endoscope is reliable and predictive of true intra-abdominal pressure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. For example, the electrode tool body may also be formed from PEEK or PTFE. Also, other transvisceral approaches could be used, such as transesophageal, transcolonic, transvaginal approaches.

It is intended that the following claims define the scope of the invention and that methods and structures within the scope of these claims and their equivalents be covered thereby.

What is claimed is:

1. A method of providing electrical stimulation to a patient's diaphragm comprising:
introducing an endoscope transviscerally into a body cavity of the patient;
delivering an electrode into the patient's body cavity through a lumen of the endoscope;
applying suction to attach the electrode to a stimulation site on the target tissue; and
delivering a stimulation pulse to the stimulation site.

2. The method of claim 1 wherein the step of delivering the electrode comprises passing an electrode tool through the endoscope lumen, the electrode tool comprising the electrode and a suction lumen, the step of applying suction comprising applying suction to the suction lumen.

3. The method of claim 2 wherein the electrode tool further comprises a handle, the step of applying suction comprising actuating a suction actuator on the handle.

4. The method of claim 2 further comprising releasing suction to detach the electrode.

5. The method of claim 4 wherein the electrode tool further comprises a handle, the releasing step comprising actuating a release actuator on the handle.

6. The method of claim 2 wherein the electrode tool further comprises a handle, the step of delivering a stimulation pulse comprising actuating a stimulating actuator on the handle.

7. The method of claim 2 further comprising using the electrode tool to mark the stimulation site with a marking agent.

8. The method of claim 7 wherein the electrode tool comprises a marking port, the step of using the electrode tool to mark the stimulation site comprising delivering a marking agent through the marking port.

9. The method of claim 7 wherein the electrode tool further comprises a handle, the step of using the electrode tool to mark the stimulation site comprising actuating a marking actuator on the handle.

10. The method of claim 2 wherein the step of introducing the endoscope comprises introducing the endoscope transgastrically.

11. The method of claim 2 wherein the stimulation site is a first stimulation site, the method further comprising moving the electrode to a second stimulation site within the body cavity after delivering a stimulation pulse to the first stimulation site and delivering a stimulation pulse to the second stimulation site.

12. A method of providing electrical stimulation to a target tissue within a patient comprising:
introducing an endoscope translumenally into a body cavity of the patient;
passing an electrode tool through the endoscope lumen, the electrode tool comprising an electrode and a marker;
placing the electrode at a stimulation site on the target tissue;
delivering a stimulation pulse to the stimulation site; and
marking the stimulation site with the electrode tool marker.

13. The method of claim 12 further comprising applying suction to the stimulation site through a suction lumen of the electrode tool after placing the electrode.

14. The method of claim 13 wherein the electrode tool further comprises a handle, the step of applying suction comprising actuating a suction actuator on the handle.

15. The method of claim 13 further comprising releasing suction to detach the electrode.

16. The method of claim 15 wherein the electrode tool further comprises a handle, the releasing step comprising actuating a release actuator on the handle.

17. The method of claim 13 wherein the electrode tool further comprises a handle, the step of delivering a stimulation pulse comprising actuating a stimulation actuator on the handle.

18. The method of claim 13 wherein the electrode tool further comprises a handle, the marking step comprising actuating a marking actuator on the handle.

19. The method of claim 12 wherein the electrode tool marker comprises a marking lumen and a marking agent port, the marking step comprising delivering a marking agent through the marking lumen and marking agent port.

20. The method of claim 12 wherein the step of introducing the endoscope comprises introducing the endoscope transgastrically.

21. The method of claim 12 wherein the stimulation site is a first stimulation site, the method further comprising moving the electrode to a second stimulation site within the body cavity after delivering a stimulation pulse to the first stimulation site and delivering a stimulation pulse to the second stimulation site.

22. An endoscopic electrode tool comprising:
a body adapted to be inserted through a working channel of an endoscope transviscerally into a patient's body cavity to a tissue stimulation site, the body comprising a suction lumen and a suction port at a distal end of the body communicating with the suction lumen, and
an electrode supported by the body at the distal end of the body, the electrode being connectable with a source of stimulation current.

23. The endoscopic electrode tool of claim 22 further comprising a handle supporting a proximal end of the electrode tool body, the handle being adapted to advance and withdraw the electrode tool from an endoscope inserted translumenally into a patient's body cavity.

24. The endoscopic electrode tool of claim 23 wherein the handle comprises a suction actuator adapted to apply suction to the suction lumen to attach the electrode to the stimulation site.

25. The endoscopic electrode tool of claim 23 wherein the handle comprises a suction release actuator adapted to release suction from the suction lumen.

26. The endoscopic electrode tool of claim 23 wherein the handle comprises a stimulation actuator adapted to apply stimulation current from the stimulation source to the electrode.

27. The endoscopic electrode tool of claim 23 wherein the electrode tool body further comprises a marking lumen communicating with a marking agent port at the distal end of the body, the marking lumen and marking agent port being adapted to deliver a marking agent to the stimulation site.

28. The endoscopic electrode tool of claim 27 wherein the handle further comprises a marking actuator adapted to deliver a marking agent through the marking lumen to the marking port.

29. The endoscopic electrode tool of claim 22 wherein the electrode tool body further comprises a marking lumen communicating with a marking agent port at the distal end of the body, the marking lumen and marking agent port being adapted to deliver a marking agent to the stimulation site.

30. The endoscopic electrode tool of claim 29 wherein the electrode surrounds the marking port.

31. The endoscopic electrode tool of claim 29 wherein the marking port, suction port and electrode are disposed on a lateral wall of the electrode tool body.

32. The endoscopic electrode tool of claim 22 wherein the electrode surrounds the suction port.

33. The endoscopic electrode tool of claim 22 further comprising a plurality of suction ports at the distal end of the body.

34. The endoscopic electrode tool of claim 33 wherein the suction ports and electrode are disposed on a lateral wall of the electrode tool body.

35. An endoscopic electrode tool comprising:
a body adapted to be inserted through a working channel of an endoscope transviscerally into a patient's body cavity to a tissue stimulation site, the body comprising a marking lumen communicating with a marking lumen port at a distal end of the body, and
an electrode supported by the body at the distal end of the body, the electrode being connectable with a source of stimulation current.

36. The endoscopic electrode tool of claim 35 further comprising a handle supporting a proximal end of the electrode tool body, the handle being adapted to advance and withdraw the electrode tool from an endoscope inserted transviscerally into a patient's body cavity.

37. The endoscopic electrode tool of claim 36 wherein the handle comprises a stimulation actuator adapted to apply stimulation current from the stimulation source to the electrode.

38. The endoscopic electrode tool of claim 36 wherein the handle comprises a marking actuator adapted to deliver a marking agent through the marking lumen to the marking port.

39. The endoscopic electrode tool of claim 35 wherein the electrode surrounds the marking port.
